(19) United States
(12) Patent Application Publication
(10) Pub. No.: US 2005/0158258 A1
(43) Pub. Date: Jul. 21, 2005

(54) Methods and compositions for the treatment of skin changes associated with aging and environmental damage

(75) Inventor: Louis B. Fisher, Dallas, TX (US)

Correspondence Address: FULBRIGHT & JAWORSKI L.L.P., 600 CONGRESS AVE., SUITE 2400, AUSTIN, TX, 78701, US

(73) Assignee: Mary Kay Inc.

(21) Appl. No.: 10/761,810

(22) Filed: Jan. 21, 2004

(51) Int. Cl. A61K 7/021; A61K 7/025; A61K 35/84; A61K 31/385; A61K 31/455

(52) U.S. Cl. 424/63; 424/64; 424/195.15; 514/356; 514/440

(57) ABSTRACT

The present invention relates to novel methods and compositions comprising a combination of ingredients for treating aged, mature, nutritionally-compromised, or environmentally-damaged skin. These methods and compositions provide improvements in the skin's visual appearance, physiological functions, clinical properties, and biophysical properties. The compositions of the present invention can include, for example, a compound that stimulates microcirculation through the skin, a compound that stimulates the immune system, a compound that reduces ultraviolet light or sun exposure damage, a compound that evens out the pigmentation of the skin, and/or a compound that improves the barrier properties of the skin.

### BACKGROUND OF THE INVENTION

A. Field of the Invention

The present invention relates generally to compositions and methods for treating aged or environmentally damaged skin. In particular, the present invention is directed towards compositions and methods for their use comprising a combination of compounds that can improve the physiological function of the skin, the metabolism of the skin, and/or the physical appearance of the skin.

B. Background of the Invention

There are a variety of changes associated with chronological skin aging (e.g., biochemical and physiological deterioration of skin cells), photoaging (e.g., due to the adverse effect of ultraviolet light on the skin), and environmentally damaged skin (e.g., due to environmental pollutants, sun light, chemicals, disease pathologies, and smoking). As these changes occur there is an increasingly obvious change in the appearance of the skin, particularly in those body sites chronically exposed to sunlight or environmental damage. With advancing age, these changes can be seen throughout the body surface.

These changes can include, for example, the appearance of fine lines and wrinkles, increased sagging, loss of elasticity, loss of firmness, and a loss of color evenness, coarse surface textures, and mottled pigmentation. Changes that are not as visually obvious, but which are increasingly noticeable with age or environmental damage, involve a decrease in the circulation of blood through the extremities and the skin. Some of the changes with age are directly related to metabolic alterations, e.g., a slowdown in the breakdown and regeneration of the dermal proteins-collagen and elastin. This can result in dermal tissue being maintained with increasingly poor function (e.g., decreasing elasticity and firmness). Other damage includes that seen in the dermal connective tissue and also the pigmentary system where some skin sites demonstrate increasing pigmentation while others demonstrate a decrease in pigmentation. A loss of Langerhans cells can also be seen with increasing sun exposure.

Several different approaches have be used to treat damaged skin caused by aging, environmental factors, chemicals, or malnutrition. One approach involves the use of specific agents to directly stimulate or inhibit selected biochemical targets. Examples include the use of retinoids to stimulate collagen and glycosaminoglycan synthesis by fibroblasts (Schiltz, et al., 1986). Another approach is to use agents or processes that stimulate the rate at which the epidermis replaces itself, a process known as epidermal cell renewal. Increases in epidermal cell renewal rates usually result from a more rapid rate of replication of epidermal basal cells, and can be caused by diverse stimuli such as chemical or physical injury, adverse environmental conditions, or direct stimulators of basal cell division.

Some examples of chemical injury include allergic responses or non-allergic contact irritation, pH extremes, or interaction of the stratum corneum with household or industrial chemicals or pollutants. Physical injury can include skin abrasion, friction (i.e. on the soles and heels of the feet), or removal of the stratum corneum by physical exfoliation (e.g., cosmetic masks) or by tape stripping. Agents that directly or indirectly stimulate basal cell division include hydroxy acids, retinoids, or barrier disrupters. For example, U.S. Pat. No. 5,720,963 discloses that a combination of hydroxy acids, retinoids, and cerebrosides causes chronic injury to the stratum corneum and results in epidermal and dermal repair of the structurally-deteriorated skin. U.S. Pat. No. 6,495,126, for example, uses a combination of surfactants and chelating agents to stimulate an endogenous stratum corneum chymotryptic proteinase that causes a loosening of corneocytes, resulting in an increased rate of epidermal replacement and chronic anti-aging benefits. Adverse environmental exposures that can result in more rapid epidermal turnover rates include UVA, UVB, and IR radiation from the sun and cold coupled with low relative humidity (i.e. low dew point).

Many of the above methods of increasing stratum corneum renewal rates have various drawbacks, such as significant irritation to the skin, skin toxicity, or low pH. In addition, most of these methods involve the invocation of chronic damage to the skin, which sets up repair mechanisms. For most of the existing treatments, there will be a period of time, up to several weeks or months, during which the skin becomes irritated and after which tolerance sets in and the symptoms of irritation may decrease and/or cease.

### SUMMARY OF THE INVENTION

The present invention overcomes deficiencies in the art by providing compositions and methods for their use that can be used to treat aged, mature, nutritionally-compromised, and/or environmentally-damaged skin.

One aspect of the present invention includes a composition comprising ximenynic acid, niacin, alpha-lipoic acid, and/or a mushroom extract, or any combination thereof, wherein the composition is formulated as a cosmetic blend. In a specific embodiment, the composition can include ximenynic acid and any one of niacin, alpha-lipoic acid, and/or a mushroom extract. The mushroom extract can be, for example, matsutake mushroom extract or shiitake mushroom extract. In a particular aspect, the composition comprises ximenynic acid, niacin, alpha-lipoic acid, matsutake mushroom extract, and shiitake mushroom extract.

The compositions of the present invention can be comprised in a cosmetic vehicle. The cosmetic vehicle can comprise an emulsion, a cream, a lotion, a solution, an anhydrous base, a gel, or an ointment, or any other vehicle known to a person of ordinary skill in the art. In particular aspects, the emulsion can be an oil-in-water emulsion or a water-in-oil emulsion. The solutions of the present invention can be, for example, an aqueous solution or hydro-alcoholic solution. The anhydrous base can be a lipstick or a powder.

In other non-limiting aspects, the compositions of the present invention can be comprised in an anti-aging product or a moisturizing product. In particular aspects, the compositions can be adapted for application at least one, two, three, four, five or more times a day during use. The compositions can also be chemically compatible.

In particular non-limiting embodiments, the compositions can comprise from about 0.001% to about 5.0% of ximenynic acid or from about 0.05% to about 1.0% of ximenynic acid. In other aspects, the compositions can comprise from about 0.0001% to about 5.0% of niacin or from about 0.0001% to about 0.5% of niacin. In still other embodiments, the compositions can comprise from about 0.001% to about 5.0% of alpha-lipoic acid, or from about 0.05% to about 1.0% of alpha-lipoic acid. In yet another aspect, the compositions can comprise from about 0.001% to about 5.0% of the mushroom extract or from about 0.001% to about 0.5% of the mushroom extract.

Also provided are methods of using the compositions described throughout the specification to treat or prevent damaged skin. A particular method of the present invention can include, for example, a method of treating or preventing damaged skin comprising topical application of a composition comprising at least two of the following: ximenynic acid, and niacin, alpha-lipoic acid, or a mushroom extract, wherein the composition is formulated as a cosmetic blend. The composition can be chemically compatible. In other aspects, the composition can be topically applied in an amount effective to improve the barrier properties of the skin, to increase the microcirculation of the skin, to stimulate the immune system, to reduce the damage caused by ultraviolet light, and/or to even out pigmentation of the skin.

“Damaged skin,” as that term is used in the specification and claims, includes aged skin, nutritionally compromised skin, or environmentally damaged skin. Environmentally damaged skin includes, for example, skin damaged by UV light, chronic sun exposure, environmental pollutants, chemicals, disease pathologies, or smoking.

In particular embodiments, there is provided a composition comprising a compound that stimulates microcirculation through the skin, a compound that stimulates the immune system, a compound that reduces ultraviolet light or sun exposure damage, a compound that evens out the pigmentation of the skin, and/or a compound that improves the barrier properties of the skin, wherein the composition is formulated as a cosmetic blend. The compound that stimulates microcirculation through the skin can be, for example, niacin or capsaicin. The compound that stimulates the immune system can be, for example, shiitake mushroom extract. The compound that reduces ultraviolet light or sun exposure damage can be, for example, alpha lipoic acid. The compound that evens out pigmentation of the skin can be, for example, matsutake mushroom extract. The compound that improves the barrier properties of the skin can be, for example, ximenynic acid.

In still another aspect of the present invention, there is provided a method for treating or preventing damaged skin comprising topical application of a composition comprising a compound that stimulates microcirculation through the skin, a compound that stimulates the immune system, a compound that reduces ultraviolet light or sun exposure damage, a compound that evens out the pigmentation of the skin, and/or a compound that improves the barrier properties of the skin, wherein the composition is formulated as a cosmetic blend. The compound that stimulates microcirculation through the skin can be, for example, niacin or capsaicin. The compound that stimulates the immune system can be, for example, shiitake mushroom extract. The compound that reduces ultraviolet light or sun exposure damage can be, for example, alpha lipoic acid. The compound that evens out pigmentation of the skin can be, for example, matsutake mushroom extract. The compound that improves the barrier properties of the skin can be, for example, ximenynic acid.

The terms “mixture,” “mix,” and “mixing” or any variants of these terms, when used in the claims and/or specification includes, stirring, blending, dispersing, milling, homogenizing, and other similar methods. The mixing of the components or ingredients of the disclosed compositions can form into a solution. In other embodiments, the mixtures may not form a solution. The compositions can also exist as undissolved colloidal suspensions.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

Aged, nutritionally-compromised, and environmentally-damaged skin affect many people in today's society. Fine lines, wrinkles, loss of elasticity, increased sagging, loss of firmness, loss of color eveness, coarse surface texture, and mottled pigmentation are just some examples of the effects of damaged skin. Previous attempts to treat damaged skin have various drawbacks ranging from skin irritation to skin toxicity. The present invention is an effective alternative to the use of hydroxy acids, retinoid compounds, or other materials currently used to treat aged or environmentally-damaged skin.

The compositions and methods of the present invention can be used, e.g., for improving the skin's visual appearance, function, and clinical/biophysical properties which have been changed by factors such as chronological age, chronic sun exposure, adverse environmental pollutants, household chemicals, disease pathologies, smoking, and malnutrition. In particular embodiments, the compositions include, e.g., a combination of ingredients that can improve microcirculation through the skin, normalize pigmentation of the skin, stimulate the local immune system, reduce the damage caused by ultraviolet light, and decrease the formation of free radicals. Examples of such ingredients and compounds include niacin, ximenynic acid, alpha lipoic acid, shiitake mushroom extract, and matsutake mushroom extract.

A. Niacin

Microcirculation of the skin can be improved by the use of niacin (vitamin B3). Niacin is known to be essential for healthy skin, a deficiency of which results in pellagra. Niacin cannot be manufactured by the human body. It is converted in the body to niacinamide, which is essential for cellular energy production. When given either by mouth or applied topically, niacin is known to produce flushing of the skin by causing vasodilation of the blood vessels. Niacin can be included in the compositions of the present invention in an amount appropriate to produce a sub-clinical degree of vasodilation, thereby improving the microcirculation in the skin. Niacin has also been shown to increase ceramide synthesis in keratinocytes, thereby improving skin barrier function.

As noted throughout the specification, other known ingredients that cause vasodilatation or increased blood flow to the skin can also be used in combination with or as a substitute for niacin. Additionally, derivatives of niacin are contemplated as being useful with the present invention. Examples of such substitutes and derivatives include, for example, methyl nicotinate, xanthinol nicotinate, capsaicin, hydergine, nicergoline, hawthorn extract, ginko biloba extract and grape skin extract.

B. Ximenynic Acid

Ximenynic acid is a conjugated, unsaturated fatty acid found in the seeds of the sandalwood. Essential unsaturated fatty acids, including omega-3 and omega-6 essential fatty acids are important to the structure and function of the stratum corneum barrier. Women of a number of African tribes use it in the form of a mask that assists with wound healing. It also reinvigorates and firms the skin. Ximenynic acid can stimulate the synthesis of the eicosanoids. It also has a direct effect on arterial smooth muscle cells, thereby increasing capillary blood flow.

As noted throughout the specification, other known ingredients that play an important role in the structure and function of the stratum corneum barrier can also be used in combination with or as a substitute for ximenynic acid. Additionally, derivatives of ximenynic acid are contemplated as being useful with the present invention. Examples of such substitutes include, for example, essential fatty acids, stearolic acid, ongokea nut extract, borage oil, evening primrose oil, spirulina extract, sunflower oil, safflower oil, flaxseed oil, walnut oil, canola oil, or soy bean oil.

C. Alpha Lipoic Acid

Alpha lipoic acid has been called the universal anti-oxidant since it is able to quench both lipid and water soluble free radicals. It is normally present in the cell in small quantities. It serves as a co-enzyme in sugar metabolism, but is also available to serve as an anti-oxidant when present in higher concentrations.

Ultraviolet light exposure results in the production of free radicals that can damage cell structures. The presence of anti-oxidants in the tissue is an important protection against the tissue damage that can occur with continued exposure to UV light, pollution, and smoking, for example. Alpha lipoic acid can be used as an effective anti-oxidant.

As noted throughout the specification, other known ingredients that have antioxidant properties can also be used in combination with or as a substitute for alpha-lipoic acid. Additionally, derivatives of alpha-lipoic acid are contemplated as being useful with the present invention. Examples of such substitutes include, for example, antioxidants that are described throughout the specification and that are known to a person of ordinary skill in the art.

D. Shiitake Mushroom Extract In Asia, the shiitake mushroom has been nicknamed the “elixir of life” (Bhosle and Vaidya, 2002). It has been shown to boost the immune system, including increasing T-cell activity. Because there is an age related decline in immune system activity and response time, exposure to noxious agents can result in a decrease in the cutaneous display. This in turn can result in more severe consequences that develop from exposure to common irritants and allergens.

Shiitake mushroom extract contains lentinane, eritadenine, amino acids, minerals, and trace elements such as potassium, sulfur, and phosphorous. Lentinane is a 1,3, beta-glucan polysaccharide fraction.

As noted throughout the specification, other known ingredients that can increase the activity or boost the immune system can also be used in combination with or as a substitute for shiitake mushroom extract. Additionally, derivatives of shiitake mushroom extract are contemplated as being useful with the present invention. Examples of such substitutes include, for example, Lentinus lepideus, L. schaefferi, L. tigrinus, Ganoderma lucidum, Grifola frondosa, and Cordyceps sinensis.

E. Matsutake (Song-Yi) Mushroom Extract (Matsutake Tricholoma)

With exposure to the environment, freckles and pigmented spots can darken and other pigmented lesions can develop. Although the degree to which this occurs varies from person to person, aging skin will invariably demonstrate a variety of pigmented spots throughout the body. The Matsutake mushroom extract can be used to remove darkened facial spots that develop due to exposure to the summer sun. Matsutake mushroom contains alpha and beta pinene, cembrene, S-matsutake alcohol, methyl cis-alpha methyl cinnamate, 2-octen-1-ol, and a variety of amino acids.

As noted throughout the specification, other known ingredients that play an important role in skin pigmentation can also be used in combination with or as a substitute for matsutake mushroom extract. Examples of such substitutes include, for example, Ganoderma lucidum, Lentinus edodes, Angelica sinensis, mulberry root bark extract, Arbutin, Licorice extract, and Scutelaria extract.

F. Source of Specific Compounds and Extracts

The specific compounds, extracts, and active ingredients in such compounds and extracts contemplated by the present invention can be obtained by any means known to a person of ordinary skill in the art. For example, the compounds, extracts, and active ingredients can be isolated by obtaining the source of such compounds and extracts. Further, the compounds, extracts, and active ingredients can be purified by any number of techniques known to a person of ordinary skill in the art. Such purification techniques include, e.g., Polyacrylamide Gel Electrophoresis, High Performance Liquid Chromatography (HPLC), Gel chromatography or Molecular Sieve Chromatography, and Affinity Chromatography.

In addition, the compounds, extracts, and active ingredients can be obtained by chemical synthesis or by recombinant means by using conventional techniques. For example, various automatic polypeptide synthesizers are commercially available and can be used in accordance with known protocols. See, for example, Stewart and Young, (1969); Tam et al., (1983); Merrifield, (1986); and Barany and Merrifield (1979), Houghten (1985). As for recombinant means, examples include the expression of a nucleic acid sequence encoding a peptide or polypeptide in an in vitro translation system or in a living cell.

G. Equivalents

Known and unknown equivalents to the specific compounds, mushroom extracts, and active components in such compounds and extracts discussed throughout this specification can be used with the compositions and methods of the present invention. The equivalents can be used as substitutes for the specific compounds, extracts, and active components. The equivalents can also be used to add to the methods and compositions of the present invention. By way of example, equivalents to niacin, alpha-lipoic acid, ximenynic acid, shiitake mushroom extract, and/or matsutake mushroom extract can be used with the methods and compositions of the present invention.

A person of ordinary skill in the art would be able to recognize and identify acceptable known and unknown equivalents to the specific compounds, extracts, and active components in such compounds and extracts without undue experimentation.

H. Compositions of the Present Invention

A person of ordinary skill would recognize that the compositions of the present invention can include any number of combinations of compounds and/or extracts, or derivatives therein. It is also contemplated that that the concentrations of the compounds and extracts can vary. In other non-limiting embodiments, for example, the compositions may include in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein of at least one of the compounds, extracts, or derivatives that are mentioned throughout the specification and claims. A person of ordinary skill in the art would understand that the concentrations can vary depending on the addition, substitution, and/or subtraction of the compounds, extracts and acceptable substitutes to these compounds and extracts.

The disclosed compositions of the present invention may also include various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

I. Cosmetic Vehicles

The present compositions are effective in all types of cosmetic vehicles. Non-limiting examples of suitable cosmetic vehicles include emulsions, creams, lotions, solutions (both aqueous and hydro-alcoholic), anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use in the present invention.

In preferred embodiments, the cosmetic vehicle is selected from oil-in-water emulsions, hydro-alcoholic solutions, or encapsulated beads in anhydrous systems. With respect to oil-in-water emulsions, such emulsions and their compositions and methods of making are well known in the art. It is important, however, that the concentrations and combinations of the compounds and extracts be selected in such a way that the combinations are chemically compatible and do not form complexes which precipitate from the finished product.

J. Cosmetic Products

The composition of the present invention can also be used in many cosmetic products including, but not limited to, moisturizing cream, skin benefit creams and lotions, gels, ointments, foundation, night cream, lipstick, cleansers, toners, masks, and/or color cosmetic products. The composition is most preferably used in anti-aging products for the face and other body parts, most especially leave-on products.

K. Additional Compounds and Agents that can be Used in Combination with the Present Compositions

Compositions of the present invention can include other beneficial agents and compounds such as, for example, acute or chronic moisturizing agents (including, e.g., humectants, occlusive agents, and agents that affect the natural moisturization mechanisms of the skin), anti-oxidants, sunscreens having UVA and/or UVB protection, skin lightening agents (e.g. hydroquinone), emollients, anti-irritants, vitamins, trace metals, anti-microbial agents, botanical extracts, fragrances, and/or dyes and color ingredients.

1. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays)oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quatemium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

2. Antioxidants

Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

3. Compounds Having Ultraviolet Light Absorbing Properties

Non-limiting examples of compounds that have ultraviolet light absorbing properties that can be used with the compounds of the present invention include benzophenone, benzophenone-1, benzophenone-2, benzophenone-3, benzophenone-4 benzophenone-5, benzophenone-6, benzophenone-7, benzophenone-8, benzophenone-9, benzophenone-10, benzophenone-11, benzophenone-12, benzyl salicylate, butyl PABA, cinnamate esters, cinoxate, DEA-methoxycinnamate, diisopropyl methyl cinnamate, ethyl dihydroxypropyl PABA, ethyl diisopropylcinnamate, ethyl methoxycinnamate, ethyl PABA, ethyl urocanate, glyceryl octanoate dimethoxycinnamate, glyceryl PABA, glycol salicylate, homosalate, isoamyl p-methoxycinnamate, PABA, PABA esters, Parsol 1789, and isopropylbenzyl salicylate.

4. Additional Compounds and Agents

Non-limiting examples of additional compounds and agents that can be used with the compositions of the present invention include skin lightening agents (e.g. kojic acid, hydroquinone, ascorbic acid and derivatives, retinoids and their derivatives, and niacinamide), emollients (e.g. esters and fatty acids), vitamins (e.g. D, E, A, K, and C), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), preservatives (e.g. BHA), emollients (i.e. organic esters, fatty acids, lanolin and its derivatives, plant and animal oils and fats, and di- and triglycerides), antimicrobial agents (e.g., triclosan and ethanol), and fragrances (natural and artificial).

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A Non-Limiting Example of One Composition of the Present Invention

A non-limiting example of one embodiment of the present invention is exhibited in Table 1. The ingredients in Table 1 were chosen because of their effects known to native peoples and because of their ability to improve the microcirculation through the skin, normalize pigmentation, stimulate the local immune system, reduce the damage caused by ultraviolet light, or decrease the formation of free radicals.

TABLE 1
A non-limiting example of specific concentrations of ingredients
used in one embodiment of the present invention

| INGREDIENT | % CONCENTRATION |
|---|---|
| Niacin | 0.015 |
| Alpha-lipoic acid | 0.5 |
| Ximenynic acid | 0.5 |
| Shiitake Mushroom Extract | 0.1 |
| Matsutake Mushroom Extract | 0.1 |

As noted above, derivatives of these ingredients can be used as substitutes. Additionally, other ingredients with similar physiological activities are contemplated as being useful as substitutes or as additional ingredients that can be used with the compositions of the present invention.

### Example 2

### The Efficacy of the Composition in Table 1 when Formulated in an Oil-in-Water Emulsion

The composition described in Table 1 was formulated into an oil-in-water emulsion. A person of ordinary skill in the art would be able to incorporate this blend (or these materials) into any type of vehicle discussed throughout the specification. Table 2, for example, includes a description of how to make an oil-in-water emulsion that includes the composition described in Table 1.

TABLE 2
A Non-limiting Vehicle formula as used in this study*

| | | |
|---|---|---|
| A | Water | 58.4 |
| A | Glycereth-26 | 5.0 |
| A | Hispagel | 5.0 |
| A | Disodium EDTA | 0.05 |
| A | Carbopol 940, 2% | 15.0 |
| B | Lecinol S-10 | 1.0 |
| C | Cosmowax J | 1.25 |
| C | Finsolve TN | 6.0 |
| C | Dimethicone | 0.5 |
| C | Isostearyl Alcohol | 1.25 |
| C | Cetyl Alcohol | 0.7 |
| C | Silica | 0.35 |
| D | Triethanolamine, 99% | 1.16 |
| D | Water | 1.60 |
| E | Germaben II | 1.0 |
| F | Sodium PCA | 0.11 |
| F | Prodew 400 | 0.7 |
| F | Tocopheryl Acetate | 0.1 |
| F | Phospholipid EFA | 0.82 |

*Procedure to make Vehicle: Add the ingredients in A to vessel, in order, at room temperature, mixing between additions. Begin heating to 75° C. At 50° C., add B. At 75° C. add C, in order, mixing between additions. As mixture cools, add D at 65° C. At 45° C., add E and F.

The effectiveness of this oil-in-water formulation was tested on twenty subjects/panelists. The twenty subjects applied the composition twice daily to the face for a period of eight weeks. As noted in Table 3, the objective results indicate an improvement in a variety of changes that are associated with aging skin.

TABLE 3
Effect of the composition in Table 1 on the human skin

| % Improvement Compared | |
|---|---|
| to Baseline | |
| | Vehicle + |
| | Composition |
| Vehicle | of Table 1 |

| Skin Benefit | Week 4 | Week 8 | Week 4 | Week 8 |
|---|---|---|---|---|
| Cheek Moisture | 20.6 | 33.5 | 40.1 | 56.4 |
| Neck Moisture | 27.9 | 36.5 | 40.1 | 60.5 |
| Firmness | 12.1 | 24.4 | 21.4 | 35.3 |
| Softness/Suppleness | 22.2 | 32.4 | 34.5 | 51.0 |
| Canthus Wrinkles | 17.2 | 28.4 | 34.9 | 55.3 |
| Clarity | 4.8 | 8.5 | 6.9 | 15.0 |
| Surface Fine Lines | 18.1 | 29.2 | 33.2 | 52.2 |
| Dryness | 32.7 | 51.0 | 40.0 | 62.8 |

The results noted in Table 3 were obtained by using objective methods which included instrumental measurements and/or expert grading systems. The results were obtained approximately 24 hours after the final application. Cheek and neck moisture was evaluated using impedance measurements with the Nova Dermal Phase Meter. Firmness was evaluated using a Hargens ballistometer, a device that evaluates the elasticity and firmness of the skin by dropping a small body onto the skin and recording its first two rebound peaks. As firmness and elasticity increase, the ratio of the magnitude of the second peak to the first will increase. Clarity was evaluated using a Minolta Chromameter, which measures the total light reflected from the skin compared to the amount of red and brown/yellow light. These measurements were mathematically analyzed to determine the clarity of the skin, as Clarity=L*/(a*²+b*²)^{1/2}. Dryness was determined by an expert grader using a calibrated visual analog scale from 1 to 10. Fine lines or surface fine lines were counted by expert graders, and the severity of the lines scored according to a modification of the Packman-Gans method (Packman and Gans, 1978). Canthus wrinkles were quantified by computer-assisted image analysis of negative Silflo replicas, and skin softness/suppleness was evaluated using the Gas Bearing Electrodynamometer, an instrument that measures the stress/strain properties of the skin.

The effectiveness of the oil-in-water formulation described in Table 1 was also measured by the panelists' self assessment. The results of the self assessment tests are noted in Table 4.

TABLE 4
Effects of the composition described in Table 1 on Panelist Self Assessment
of Their Skin Condition During an 8-Week Treatment Period

| | % of Panelists Perceiving Improved Skin Condition* | | | | | |
|---|---|---|---|---|---|---|
| | Vehicle | | | Patent blend in Vehicle | | |
| Skin Condition | 2 Weeks | 4 Weeks | 8 Weeks | 2 Weeks | 4 Weeks | 8 Weeks |
| Dryness | 53.3 | 66.7 | 86.7 | 60.00 | 80.00 | 100.0 |
| Smoothness | 46.7 | 60.0 | 80.0 | 60.0 | 80.0 | 93.3 |
| Lines and | 6.7 | 26.7 | 60.0 | 26.7 | 60.0 | 80.0 |
| Wrinkles | | | | | | |
| Firmness | 6.7 | 46.7 | 66.7 | 26.7 | 66.7 | 86.7 |
| Softness | 33.3 | 46.7 | 73.3 | 60.0 | 66.7 | 86.7 |
| Healthy | 13.3 | 26.7 | 46.7 | 26.7 | 40.0 | 80.0 |
| Glow | | | | | | |
| Elasticity | 26.7 | 53.3 | 66.7 | 33.3 | 73.3 | 93.3 |
| Looks | 13.3 | 46.7 | 73.3 | 26.7 | 66.7 | 93.3 |
| Younger | | | | | | |
| Looks | 20.0 | 46.7 | 80.00 | 26.7 | 66.7 | 86.7 |
| Healthier | | | | | | |
| *Twenty panelists in each of the treatment cells participated in the study. After 2, 4, and 8 weeks of product use, the panelists rated their skin condition on a 5-point scale as compared to the condition at the start of the study. The scale ranged from the assessed parameter being much less improved, somewhat less | | | | | | |
| # improved, no change, somewhat greater improved, and much greater improved. The values represent the percent of panelists who perceived improvement at the given point in time. | | | | | | |

All of the compositions and/or methods disclosed and claimed in this specification can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,720,963
U.S. Pat. No. 6,495,126
Barany and Merrifield, In: The Peptides, Gross and Meienhofer (Eds.), Academic Press, NY, 1-284, 1979.
Bhosle S. R. and Vaidya J. G.: SHIITAKE, MUSHROOM OF 21st CENTURY. Elixir of Life Publ. Satyajeet Prakashan 2002.
Houghten, Proc. Natl. Acad. Sci. USA, 82(15): 5131-5135, 1985.
Merrifield, Science, 232(4748): 341-347, 1986.
Packman-Gans, J Soc. Cosmetic Chem., 29: 70, 1978.
Remington's Pharmaceutical Sciences, 18th Ed. Mack Printing Company, 1990.
Schiltz et al. J Investigative Dermatology, 87: 663-667, 1986.
Stewart and Young, In: Solid Phase Peptide Synthesis, 24-66, Freeman, San Francisco, 1969.
Tam et al., J. Am. Chem. Soc., 105: 6442, 1983.

1. A composition comprising at least two of the following:
(a) ximenynic acid; and
(b) niacin, alpha-lipoic acid, or a mushroom extract,
wherein the composition is formulated as a cosmetic blend.

2. The composition of claim 1, wherein the composition is comprised in a cosmetic vehicle.

3. The composition of claim 2, wherein the cosmetic vehicle comprises an emulsion, a cream, a lotion, a solution, an anhydrous base, a gel, or an ointment.

4. The composition of claim 3, wherein the emulsion is an oil-in-water emulsion or a water-in-oil emulsion.

5. The composition of claim 3, wherein the solution is an aqueous solution or hydro-alcoholic solution.

6. The composition of claim 3, wherein the anhydrous base is a lipstick or a powder.

7. The composition of claim 1, wherein the composition is comprised in an anti-aging product or a moisturizing product.

8. The composition of claim 1, wherein the composition is adapted for application at least once a day during use.

9. The composition of claim 1, wherein the composition is adapted for application at least twice a day during use.

10. The composition of claim 1, wherein the composition comprises from about 0.001% to about 5.0% of ximenynic acid.

11. The composition of claim 1, wherein the composition comprises from about 0.0001% to about 5.0% of niacin.

12. The composition of claim 1, wherein the composition comprises from about 0.001% to about 5.0% of alpha-lipoic acid.

13. The composition of claim 1, wherein the composition comprises from about 0.001% to about 5.0% of the mushroom extract.

14. The composition of claim 1, wherein the composition is chemically compatible.

15. The composition of claim 1, wherein mushroom extract is matsutake mushroom extract or shiitake mushroom extract.

16. The composition of claim 15, wherein the composition comprises:
(a) ximenynic acid;
(b) niacin;
(c) alpha-lipoic acid;
(d) matsutake mushroom extract; and
(e) shiitake mushroom extract.

17. A method of treating or preventing damaged skin comprising topical application of a composition comprising at least two of the following:
(a) ximenynic acid; and
(b) niacin, alpha-lipoic acid, or a mushroom extract,
wherein the composition is formulated as a cosmetic blend.

18. The method of claim 17, wherein the composition is topically applied in an amount effective to improve the barrier properties of the skin.

19. The method of claim 17, wherein the composition is topically applied in an amount effective to increase the microcirculation of the skin.

20. The method of claim 17, wherein the composition is topically applied in an amount effective to stimulate the immune system.

21. The method of claim 17, wherein the composition is topically applied in an amount effective to reduce the damage caused by ultraviolet light.

22. The method of claim 17, wherein the composition is topically applied in an amount effective to even out pigmentation of the skin.

23. The method of claim 17, wherein the damaged skin comprises aged skin, nutritionally compromised skin, or environmentally damaged skin.

24. The method of claim 23, wherein the environmentally damaged skin comprises skin damaged by UV light, chronic sun exposure, environmental pollutants, chemicals, disease pathologies, or smoking.

25. The method of claim 17, wherein mushroom extract is matsutake mushroom extract or shiitake mushroom extract.

26. The method of claim 25, wherein the composition comprises:
(a) ximenynic acid;
(b) niacin;
(c) alpha-lipoic acid;
(d) matsutake mushroom extract; and
(e) shiitake mushroom extract.

27. A composition comprising:
(a) a compound that stimulates microcirculation through the skin;
(b) a compound that stimulates the immune system;
(c) a compound that reduces ultraviolet light or sun exposure damage;
(d) a compound that evens out the pigmentation of the skin; and
(e) a compound that improves the barrier properties of the skin,
wherein the composition is formulated as a cosmetic blend.

28. The composition of claim 27, wherein the compound that stimulates microcirculation through the skin is niacin or capsaicin, the compound that stimulates the immune system is shiitake mushroom extract, the compound that reduces ultraviolet light or sun exposure damage is alpha lipoic acid, the compound that evens out pigmentation of the skin matsutake mushroom extract, and the compound that improves the barrier properties of the skin is ximenynic acid.
